(19) United States
(12) Plant Patent Application Publication
(10) Pub. No.: US 2019/0014703 P1
(43) Pub. Date: Jan. 10, 2019

(54) Lupinus 'LSTRE04-0'

(50) ;

(71) Applicant: Kazunori Sato, Tokyo (JP)

(72) Inventor: Kazunori Sato, Tokyo (JP)

(21) Appl. No.: 15/998,132

(22) Filed: Jul. 5, 2018

Related U.S. Application Data
(60) Provisional application No. 62/604,475, filed on Jul. 8, 2017.

(51) Int. Cl. A01H 6/54

(52) U.S. Cl. PLT/263.1

(57) ABSTRACT

A new and distinct Lupinus cultivar named ‘LSTRE04-0’ is disclosed, characterized by distinctive light red flower coloration. Plants uniquely bloom under both short day and long day conditions without vernalization. The new cultivar is a Lupinus, suitable for ornamental purposes.

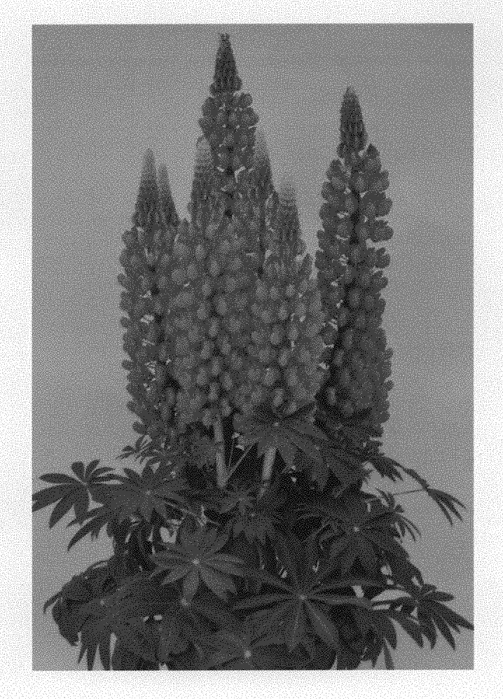

Latin name of the genus and species: Lupinus hybrida.

Variety denomination: ‘LSTRE04-0’.

### BACKGROUND OF THE INVENTION

[symbol] new variety, hereinafter referred to as ‘LSTRE04-0’, was discovered as part of a planned breeding program. The intent of the breeding program was to develop Lupine hybrids for commercial ornamental applications.

### SUMMARY OF THE INVENTION

The cultivar ‘LSTRE04-0’ has not been observed under all possible environmental conditions. The phenotype may vary somewhat with variations in environment such as temperature, day length, and light intensity, without, however, any variance in genotype. The following traits have been repeatedly observed and are determined to be the unique characteristics of ‘LSTRE04-0’These characteristics in combination distinguish ‘LSTRE04-0’ as a new and distinct Lupinus cultivar:

1. Plants uniquely bloom under both short day and long day conditions without vernalization.

2. Light red flower coloration.

### COMPARISON TO PARENT VARIETY

‘LSTRE04-0’ is similar in most horticultural characteristics to the seed parent variety. Plants of the new cultivar ‘LSTRE04-0’ however, differ in the following characteristics:

1. The new variety has a different flower color.

‘LSTRE04-0’ is similar in most horticultural characteristics to the pollen parent variety. Plants of the new cultivar ‘LSTRE04-0’ however, differ in the following characteristics:

1. The new variety has a different flower color.

### COMMERCIAL COMPARISON

‘LSTRE04-0’ can be compared to the commercial variety Lupinus ‘Beefeater’, unpatented. Plants of the new cultivar ‘LSTRE04-0’ are similar to plants of ‘West Country Purple Swirl’ in most horticultural characteristics, however, plants of the new cultivar ‘LSTRE04-0’ differ in the following:

1. Flower color of the new variety is light red, flower color of this comparator is light red with a yellow banner, as well as a tendency to tarn purplish.
2. Plants of the new variety are shorter and sturdier.
3. Plants of the new variety will begin flowering without vernalization, as required by this comparator.

‘LSTRE04-0’ can be compared to the commercial variety Lupinus ‘West Country Persian Slipper’ unpatented. Plants of the new cultivar ‘LSTRE04-0’ are similar to plants of ‘West Country Towering Inferno’ in most horticultural characteristics, however, plants of the new cultivar ‘LSTRE04-0’ differ in the following:

1. Flower color of the new variety is light red, flowers of this comparator are light red with a yellow-orange banner.
2. Plants of the new variety are shorter and sturdier.
3. Plants of the new variety will begin flowering without vernalization, as required by this comparator.

### BRIEF DESCRIPTION OF THE PHOTOGRAPH

The accompanying photograph in illustrates in full color a typical plant of ‘LSTRE04-0’ grown in a greenhouse in Santa Paula, Calif. The photograph was taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Colour Chart 2007 except where general terms of ordinary dictionary significance are used. The following observations and measurements describe ‘LSTRE04-0’ plants grown outdoors in Santa Paula, Calif. Plants are approximately 1 year old, in a 2-gallon nursery container. Measurements and numerical values represent averages of typical plant types.
Botanical classification: Lupinus hybrida ‘LSTRE04-0’.

### PROPAGATION

Typically by shoot tissue culture:
Root description: Single main root, moderate branching of short lateral roots. Cream to tan in color, not accurately measured with an R.H.S. chart.

### PLANT

Growth habit: Compact and herbaceous vigorous perennial. Mounding foliar base topped with upright raceme inflorescences.
Height: Approximately 25 cm to top of foliar plane, approximately 65 cm to top of flowering plane.
Plant spread: Approximately 40 cm.
Branching characteristics: No true branching. Leaves emerge direct from base of plant, or infrequently occur directly on the flowering stem.
Age of plant described: Approximately 1 year.

### FOLIAGE

Leaf: Palmately compound. Occurring mainly as basal whorls. Infrequently alternate along flowering stein.
   Shape.—Orbicular.
   Length.—12.0 cm.
   Diameter.—13.0 cm.
   Quantity.—Approximately 20 to 30 basal leaves, per plant. Average range 2 to 4 floral stem leaves, per stem.
Leaflets:
   Quantity.—Average range 10 to 11.
   Shape.—Oblanceolate.
   Arrangement.—Radial.
   Average length.—Longest leaflet per leaf, average 7.5 cm. Shortest leaflet per leaf average 4.5 cm.
   Average width.—Central leaflet average 2.0 cm.
   Apex.—Acute to nearly acuminate.
   Base.—Attenuate.
   Margin.—Entire.
   Texture of top surface.—Glabrous.
   Texture of bottom surface.—Glabrous.
   Appearance of top surface.—Matte.
   Appearance of bottom surface.—Matte.
   Aspect.—Flat to very slightly reflexed downward.
   Color.—Upper side: Near RHS Green 137A. Under side: Near RHS Green 137D.
   Venation.—Type: Pinnate. Venation color upper side: Near RHS Yellow-Green 145A. Venation color under side: Near RHS Yellow-Green 145A.
   Petiole.—Length: Average range 10.0 to 12.0 cm. Diameter: 0.6 cm. Color: Near RHS Yellow-Green 144D. Texture: Minutely hirsute.

### FLOWER

Natural flowering season: Spring through Summer.
Inflorescence type and habit: Single large papilonaceous flowers evenly and symmetrically arranged on racemes.
Flower longevity on plant: Individual flowers approximately 15 days on the plant, not persistent.
Quantity of flowers: 60 to 130 flowers and buds per inflorescence. Typically 4 to 6 inflorescences per plant at one time.
Inflorescence size:
   Diameter.—Approximately 8.0 cm.
   Height.—Average range 20 to 45 cm.
Flower size (excluding pedicel):
   Length.—Approximately 1.9 cm.
   Height.—Average range 2.0 cm.
Peduncle:
   Length.—Approximately 7.0 cm from uppermost leaf to lowermost flower.
   Diameter.—Approximately 0.9 cm.
   Color.—Near RHS Yellow-Green 145A, flushed Greyed-Purple 187B.
   Strength.—Flexible, very strong.
   Texture.—Short pubescence.
Pedicel:
   Length.—Approximately 1.5 cm.
   Diameter.—Approximately 0.2 cm.
   Color.—Near RHS Greyed-Purple 186B.
   Strength.—Strong.
   Texture.—Softly pubescent.
Petals:
   Quantity and arrangement.—1 upper banner, 2 lateral “wings” and a lower keel.
Banner:
   Length.—Approximately 2.0 cm.
   Width (furled, natural state).—Approximately 1.0 cm.
   Shape.—Orbicular, when unfurled.
   Margin.—Entire.
   Apex.—Mucronate.
   Base.—Obtuse.
   Texture.—Smooth, glabrous all surfaces.
   Aspect.—Folded, margins furled.
Banner color: When opening: Outer surface: Base near RHS White N155C remaining surface near Red-Purple 61C. Inner surface: Base near RHS White N155C remaining surface near Greyed-Purple 186C. Fully opened: Outer surface: Base near RHS White 155D, remaining surface near Red 53A. Inner surface: Base near RHS White N155D, remaining surface near Red-Purple 64A. Fading: Outer surface: Base near RHS White N155D, remaining surface near Red-Purple 70A and 70C. Inner surface: Base near RHS White N155D, remaining surface Red-Purple 70A and 70C.
Wing:
   Length.—Approximately 1.7 cm.
   Width).—Approximately 1.3 cm.
   Shape.—Ovate.
   Margin.—Entire.
   Apex.—Rounded, frequently fused to opposite wing at apex.
   Base.—Truncate.
   Texture.—Smooth, glabrous all surfaces.
   Aspect.—Inwardly cupped.
Wing color: When opening: Outer surface: Near RHS Red 53C. Inner surface: Near RHS Red 53C. Fully opened: Outer surface: Near RHS Red 53D. Inner surface: Near RHS Red 53D. Fading: Outer surface: Near RHS Red 52A. Inner surface: Near RHS Red 52A.
Keel:
   Length.—Approximately 1.2 cm.
   Width).—Approximately 0.6 cm.
   Shape.—Scythe.
   Margin.—Entire.
   Apex.—Awn-like.
   Base.—Truncate.
   Texture.—Smooth, glabrous all surfaces.
   Aspect.—Folded.
Keel color: When opening: Outer surface: Near RHS Red 52A. Inner surface: Near RHS Red 52A. Fully opened: Outer surface: Near Red 52B. Inner surface: Near Red 52A. Fading: Outer surface: Near Red 52C. Inner surface: Near RHS Red 52C.
Bud:
   Shape.—Flattened ovate.
   Length.—Approximately 1.5 cm.
   Diameter.—Approximately 1.0 cm.
   Color.—Lower side near RHS Red 52A blushed 53A, upper side near Red-Purple N57C.
Sepals:
   Quantity.—2, one fully developed, one minute(less than 1 mm in length), sometimes missing.
   Length.—Approximately 5 mm.
   Width.—Approximately 4 mm.
   Shape.—Deltate.
   Aspect.—Cupped.
   Apex.—Mucronate.
   Margin.—Entire. Color. Interior Surface: RHS Yellow-Green 145C. Exterior Surface: RHS Yellow-Green 145D.
   Texture.—Glabrous.
Fragrance: Strong, sweet scent.

### REPRODUCTIVE ORGANS

Stamens:
   Number.—Approximately 8.
   Filament length.—Approximately 0.9 cm.
   Filament color.—Near RHS Yellow-Green 145D.
Anthers:
   Length.—0.3 cm.
   Shape.—Elongated ovoid.
   Color.—Near RHS Yellow-Orange 15B.
   Pollen.—Scant, colored near Yellow 13C.
Pistil:
   Number.—1.
   Length.—Approximately 1.5 cm.
   Stigma.—Shape: Globular. Color: Near RHS Yellow-Orange 15B.
   Ovary.—Near RHS Green 143D, approximately 2 mm in diameter.

### OTHER CHARACTERISTICS

Disease and pest resistance: Not observed to be susceptible nor resistant to normal diseases and pests of Lupinus lactiflora x lutea.
Temperature tolerance: Typically USDA Zones 3 to 9.
Fruit/seed production: Not observed.

What is claimed is:

1. A new and distinct cultivar of Lupinus plant named ‘LSTRE04-0’ as herein illustrated and described.
